(19) United States
(12) Patent Application Publication
(10) Pub. No.: US 2021/0354096 A1
(43) Pub. Date: Nov. 18, 2021

(54) System for Controlling an Emulsification Process

(71) Applicant: Institute of Technology Sligo, Sligo (IE)

(72) Inventors: Saritha Unnikrishnan, Caltragh (IE); John Donovan, Moycullen (IE); David Tormey, Ballinafad (IE)

(21) Appl. No.: 17/286,517

(22) PCT Filed: Oct. 21, 2019
(86) PCT No.: PCT/EP2019/078599

§ 371 Date: Apr. 19, 2021

(30) Foreign Application Priority Data
Oct. 19, 2018 (GB) 1817089.4

(51) Int. Cl. B01F 3/08; B01F 15/00; A61K 9/107; G01N 15/02; G01N 15/14; G06K 9/46; G06K 9/62; G06T 7/00

(52) U.S. Cl. CPC B01F 3/0811; B01F 15/00207; B01F 15/00285; A61K 9/107; G01N 15/0227; G06T 2207/10056; G06K 9/4642; G06K 9/6247; G06T 7/0004; B01F 2215/0032; G01N 2015/1486; G01N 15/1434

(57) ABSTRACT

A system for controlling an emulsification process including the steps of acquiring images such as micrographs of an emulsification process at preset intervals between a start and an end of the emulsification process; detecting selected droplet characteristics such as size and count using image segmentation such as a histogram-based technique; analyzing the measured droplet characteristics; comparing the measured droplet characteristics with a desired droplet characteristic specification; and terminating the emulsification process when said desired droplet characteristic is achieved.

### CROSS REFERENCE TO RELATED APPLICATIONS

This application is a national phase to PCT Application No. PCT/EP2019/078599 filed Oct. 21, 2019, which in turn claims priority to UK Patent Application No. GB1817089.4 filed Oct. 19, 2018, all said applications incorporated in their entirety herein by reference thereto.

### FEDERALLY SPONSORED RESEARCH AND DEVELOPMENT

None.

### BACKGROUND OF THE INVENTION

### 1. Field of the Invention

This invention relates to an emulsification process and the control thereof.

### 2. Background

Product quality assessment, during emulsification, has been identified as a challenging task in chemical industries. Emulsions are multi-phase colloidal dispersions obtained by mixing two or more immiscible fluids. One example of an emulsion is a two-phase oil in water emulsion (o/w) which is formed when oil gets dispersed as tiny droplets (dispersed phase) in water (continuous phase). Emulsification involves breaking an immiscible fluid (e.g., oil) into a homogenous distribution of microscopic droplets. The quality of the final emulsion product is highly dependent on the droplet size distribution, which is influenced by the operating conditions and process parameters.

Currently, the quality evaluation of emulsion products in for example the pharma sector is entirely based on manual examination of samples under the microscope. This manual technique has shown high subjectivity, low repeatability and reproducibility and is remarkably slow. The existing techniques which require physical sampling can lead to significant measurement errors causing inaccurate quality assessment. This can lead to over-processing, excessive energy utilization, wastage of resources and increased cost of production.

Other emulsion quality evaluation techniques have been proposed such as laser diffraction, spectroscopic, etc., however, with these techniques dilution of samples is essential. Such techniques add additional complexity to industrial processes, are time consuming and unreliable.

All of the above-mentioned techniques are difficult to automate for the reasons hereinbefore discussed.

There have been several machine vision techniques reported in the literature for the segmentation and analysis of optical microscopic images of emulsions. Zhou et al have conducted a critical review of various image processing approaches in real-time crystal size measurements. ImageJ and Matlab are the most popular and widely used machine vision software for automated droplet detection in emulsions according to previous research. Maaβ et al have developed a machine vision tool in Matlab for automated counting and measurement of particles/droplets in multiphase systems including emulsions. However, the previous methods are limited to circular particles and Maaβ et al have proposed a future extension of the method to additionally detect irregular shapes as well.

The droplet detection techniques previously referenced have used machine vision software mainly to identify the droplet border on the phase transition between oil and water using edge detection.

In addition to that, there has been no previous research published so far on a progressive analysis of droplet characteristics other than droplet diameter during emulsification.

One object of the present invention is to provide an improved system for controlling an emulsification process and overcome the problems encountered heretofore.

### SUMMARY OF THE INVENTION

According to one aspect of the invention, there is provided a system comprising an apparatus and a method for controlling emulsification of at least two immiscible fluids.

According to another aspect of the invention, there is provided an apparatus for controlling emulsification comprising:
an imaging device configured to obtain an image of at least one droplet at pre-set intervals between a start and an end of the emulsification;
an image processor configured to detect and measure at least one droplet characteristic;
means for analyzing the measured droplet characteristics;
means for comparing the measured droplet characteristics with a desired droplet characteristics specification; and
a control switch configured to terminate the emulsification when a signal is received indicating that said desired droplet characteristics are achieved.

In one embodiment of the invention, the imaging device is adapted for mounting in situ where the emulsification process is taking place. One advantage of having the imaging device in situ where emulsification is taking place is that no sampling, manual or otherwise, is required.

In one embodiment of the invention, the imaging device is an optical imaging device.

In a further embodiment of the invention, the optical imaging device is configured to perform confocal microscopy, line confocal microscopy, deconvolution microscopy, spinning disk microscopy, multi-photon microscopy, planar illumination microscopy, Bessel beam microscopy, differential interference contrast microscopy, phase contrast microscopy, epifluorescent microscopy, bright field microscopy, dark field microscopy, oblique illumination or a combination thereof.

In a preferred embodiment of the invention, the optical imaging device is configured to perform bright field microscopy.

In another embodiment of the invention, the imaging device comprises a charge-coupled device (CCD) camera.

In another embodiment of the invention, the imaging device comprises an endoscope.

In another embodiment of the invention, the imaging device comprises a soft sensor.

In another embodiment of the invention, the image obtained by the imaging device is a micrograph.

In another embodiment of the invention, the micrograph is calibrated to a micrometer scale.

In another embodiment of the invention, the image processor is configured to detect the droplet from the pixels in the image.

In another embodiment of the invention, the image processor is configured to detect the droplets comprise a means for image segmentation.

In another embodiment of the invention, the means for image segmentation is an edge and symmetry filter.

In another embodiment of the invention, the means for image segmentation comprises a histogram-based technique.

In another embodiment of the invention, the image processor configured to detect at least one of the droplet characteristics comprises means to prepare a graphical representation from the pixels in the images.

In a preferred embodiment of the invention, the graphical representation is an edge and symmetry graphic.

In a most preferred embodiment of the invention, the graphical representation is a histogram.

In another embodiment of the invention, the image processor comprises means to calculate the mean pixel intensity from the graphical representation.

In another embodiment of the invention, the apparatus comprises means to threshold the images.

In another embodiment of the invention, the apparatus comprises means to convert the thresholded images to binary.

In another embodiment of the invention, the apparatus comprises means to apply watershed segmentation to the images.

In another embodiment of the invention, the image processor comprises means to convert the images to 8-bit.

In another embodiment, the means to convert the images to 8-bit is positioned after the imaging device and before the means to prepare a graphical representation.

In a preferred embodiment, the means to convert the images to 8-bit is positioned after the imaging device and before the means for image segmentation.

In another embodiment of the invention, the means for comparing the measured droplet characteristics with the desired droplet characteristics specification determines an emulsification quality category of the emulsion at a given pre-set interval during emulsification.

It is to be appreciated that the emulsification quality category is determined from images obtained by the image processor at pre-set intervals during emulsification.

In another embodiment of the invention, the means for comparing the measured droplet characteristics obtained from the image with a desired droplet characteristics specification involves a supervised classification model.

In a further embodiment of the invention, the supervised classification model utilizes an algorithm.

In a preferred embodiment of the invention, the algorithm of the supervised classification model adapts in response to new droplet characteristics data.

One advantage of an algorithm that adapts in response to new droplet characteristics data is that the apparatus becomes capable of machine learning and can optimize operational performances and better control emulsification based on the new data received and avoid overprocessing.

In a more preferred embodiment of the invention, the algorithm is principal component analysis (PCA) based.

In another embodiment of the invention, the apparatus comprises a central processing unit configure to control operation of the apparatus.

In a further embodiment of the invention, the central processing unit monitors the emulsion quality category at the pre-set intervals between the start and the end of emulsification to determine when the desired droplet characteristics are achieved.

In another embodiment of the invention, the apparatus comprises an alerting means for indicating the emulsion quality category of the images obtained by the image processor to the central processing unit corresponding to a quality of the emulsion at the pre-set intervals between the start and the end of emulsification.

In a further embodiment of the invention, the central processing unit sends the signal to the control switch to terminate the emulsification.

It is to be appreciated that the signal to terminate is sent once the central processing unit has determined that the desired droplet characteristics are met.

One advantage of the features as hereinbefore described is that real-time deployment of emulsion quality evaluation is enabled.

Another advantage of the features as hereinbefore described is that emulsion quality evaluation techniques can be automated.

Another advantage of the features as hereinbefore described is that in-line deployment of emulsion quality evaluation in industrial process is made possible.

According to another aspect of the invention, there is provided a method for controlling emulsification of at least two immiscible fluids including the steps:
acquiring images of at least one droplet at pre-set intervals between a start and an end of the emulsification;
detecting and measuring at least one droplet characteristic from the acquired images;
analyzing the measured droplet characteristics;
comparing the measured droplet characteristics with a desired droplet characteristics specification; and
terminating the emulsification when said desired droplet characteristics are achieved.

In one embodiment of the invention, the method includes acquiring images of the emulsification at five minute intervals between a start and an end of the emulsification.

In one embodiment of the invention, the detecting and measuring utilizes a means for image segmentation.

In another embodiment of the invention, each image obtained by the imaging device is a micrograph and the method comprises calibrating the micrograph to a micrometer scale.

In another embodiment of the invention, the method comprises converting the images to 8-bit.

In a preferred embodiment of the invention, conversion of the images to 8-bit occurs after the images have been acquired by the imaging device and before the detecting and measuring of the droplets.

In another embodiment of the invention, the method comprises preparing a graphic representation of the image from the pixels in the images.

In another embodiment of the invention, the method comprises the preparation of the graphic representation wherein the graphic representation is an edge and symmetry graphic.

In a preferred embodiment of the invention, the method comprises the preparation of the graphic representation wherein the graphic representation is a histogram.

In another embodiment of the invention, the method comprises calculating the mean pixel intensity from the graphic representation.

In another embodiment of the invention, the method comprises thresholding the images using the calculated intensity value.

In another embodiment of the invention, the method comprises converting the thresholded images to binary.

In another embodiment of the invention, the method comprises applying watershed segmentation.

In another embodiment of the invention, the method comprises categorizing the emulsion into one of several pre-defined emulsion quality categories.

In a preferred embodiment of the invention, the categorizing is determined by the comparison of the measured droplet characteristics against a desired droplet characteristics specification.

In another embodiment of the invention, the method comprises processing data on the emulsion quality category obtained from the pre-set intervals between the start and the end of emulsification to determine when the desired droplet characteristics are achieved.

In a further embodiment of the invention, the method comprises sending a signal from the central processing unit to a control switch to terminate the emulsification when the desired droplet characteristics are achieved.

In another embodiment, the invention provides a system for controlling an emulsification process including the steps:
acquiring micrographs of an emulsification process at preset intervals between a start and an end of the emulsification process;
detecting droplet size using a histogram-based technique;
analyzing the measured droplet size and count;
comparing the measured droplet size and count with a desired droplet size and count specification; and
terminating the emulsification process when said desired droplet size and count is achieved.

In another embodiment of the invention, the system includes:
calibrating the micrograph to a micrometer scale;
converting the images to 8-bit;
preparing a histogram from the pixels in the images;
calculating the mean pixel intensity;
thresholding the images using the calculated intensity value; and
converting to binary and applying watershed segmentation.

In another embodiment of the invention, the system includes:
acquiring micrographs of the emulsification process at five-minute intervals between a start and an end of the emulsification process.

### BRIEF DESCRIPTION OF THE DRAWINGS

The invention will be more clearly understood by the following description of some embodiments thereof, given by way of example only, with reference to the accompanying drawings.

FIG. 1 is a schematic representation of apparatus according to the invention for controlling an emulsification process.

FIG. 2 is a flowchart illustrating a method according to a second aspect of the invention for controlling an emulsification process.

FIG. 3 is a flowchart alongside a schematic showing a portion of the method according to FIG. 2, in particular how the droplets are detected and the input image is processed to result in an output image prior to analysis.

FIG. 4 is a set of bright field micrographs at 40× magnification obtained after a) 5 minutes b) 10 minutes c) 15 minutes d) 20 minutes e) 25 minutes f) 30 minutes of the emulsification processing.

FIG. 5 is an edge and symmetry-based image segmentation graphic.

FIG. 6 is a histogram-based image segmentation graphic in accordance with the present invention.

FIG. 7 is an input image of an emulsion micrograph (a) and a resulting output image of the detected droplets after application of HBT (b).

FIG. 8 is a comparison of output images after application of image segmentation ESF (b) and HBT (c) of an input image of an emulsion micrograph (a).

FIG. 9 is a box plot of the average droplet area (a) and a box plot of the average Feret diameter (b) obtained after ESF image segmentation.

FIG. 10 is a boxplot showing the evolution of droplet count from 5 to 30 minutes of emulsification using ESF data.

FIG. 11 is a series of box plots showing mean droplet characteristics obtained from the HBT: a) area, b) perimeter, c) Feret, d) MinFeret, e) Droplet count.

FIG. 12 is a close-up view of FIG. 11(a) and FIG. 11(c) showing box plots of average droplet area (a) and average Feret diameter (b) obtained from HBT.

FIG. 13 is a close-up view of FIG. 11(e) demonstrating the evolution of droplet count from 5 to 30 minutes of emulsification using HBT.

FIG. 14 is a comparative box plot showing the oil concentration from 5 to 30 minutes of emulsification comparing edge and symmetry detection (a) and histogram-based detection (b).

FIG. 15 is a scree plot showing the percentage variance between five principal components (PCs).

FIG. 16 is a principal component analysis score plot using the first three PCs of FIG. 15.

FIG. 17 is a linear discriminant analysis classification presented by the three discriminant functions a) LD1, b) LD2 and c) LD3.

FIG. 18 is a scatter plot showing TAMU classification as presented by LD2 vs LD1.

FIG. 19 is a confusion matrix of PC-LDA model from 5-fold cross-validation, representing the sum of five confusion matrices from the five models.

### DETAILED DESCRIPTION OF THE INVENTION

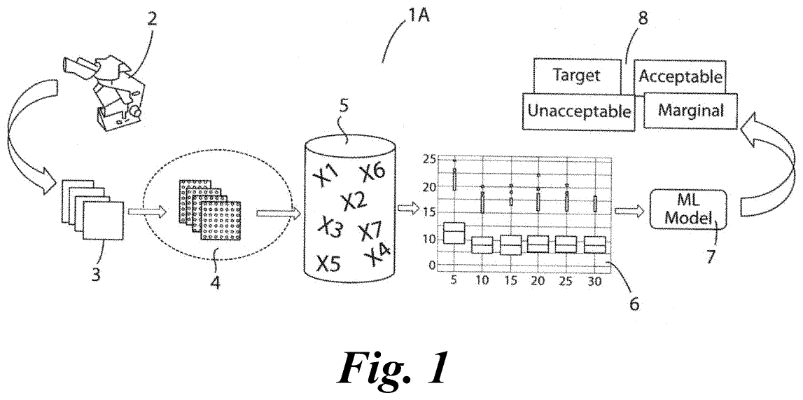

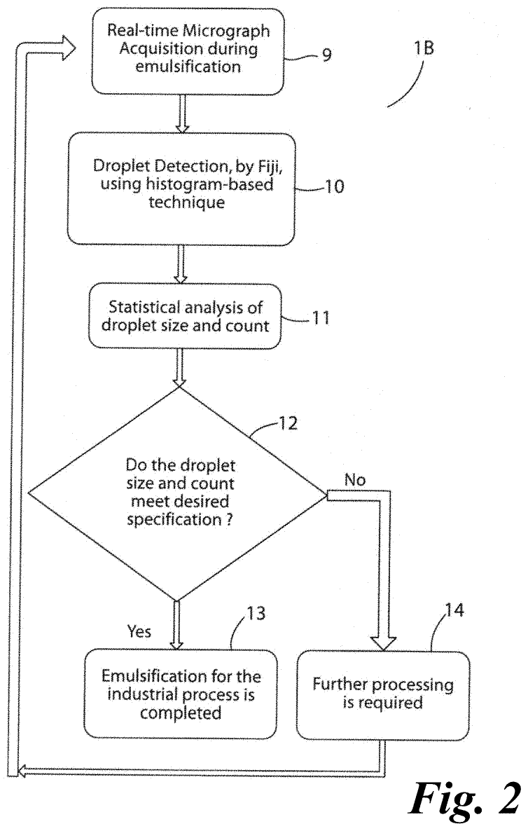

Referring to the figures, and initially to FIGS. 1 and 2, there is illustrated a system according to the invention indicated generally by the reference numeral 1, the system 1 comprising apparatus according to the invention, generally indicated by the reference numeral 1A, and a method according to the invention generally indicated by reference numeral 1B, for controlling emulsification of at least two immiscible fluids.

Referring now to FIG. 1, there is illustrated an apparatus 1A according to the invention for controlling the emulsification. The apparatus 1A comprises an imaging device 2 for the acquisition of images 3 from an emulsion. In this embodiment the imaging device is a microscope. The acquired images 3 are then processed by an image processor 4 to produce measured droplet characteristics data 5 of said droplets contained within the emulsion. A means for analyzing 6 the data 5 plots the data 5 using a graph and statistical analysis software package (e.g., graph plod prism) [not shown], a means to compare (6, 7) the data 5 against a desired droplet characteristic specification 7.

In this embodiment, the apparatus 1A comprises a means for categorizing the emulsion into an emulsion quality category 8. This categorization is based on the measured droplet characteristics data 5 as compared with the desired droplet characteristics specification 7.

It is to be appreciated that the desired droplet characteristics specification 7 is pre-determined prior to use in the system (1A; 1B).

The emulsion quality category 8 determines whether or not the desired droplet characteristics specification 7 is achieved. When the desired droplet characteristics specification 7 is achieved emulsification is terminated.

Referring now to FIG. 2 there is illustrated a method for controlling the emulsification. The method 1B includes an initial step 9 comprising real-time image acquisition during emulsification. The images are acquired at pre-set intervals; say every 5 minutes, between a start and an end of the emulsification. In this embodiment, the images are shown as micrographs. This is followed by a detection step 10 in which droplet characteristics are detected using an image segmentation technique. In this embodiment a histogram-based technique (HBT) is shown as the image segmentation technique used. Then, in an analysis step 11, the measured droplet characteristics are analyzed. In this embodiment, the droplet characteristics of interest relate to size and count. In a comparison step 12 the measured droplet characteristics are compared with a desired droplet characteristics specification. If the desired droplet characteristics specification is achieved, then the emulsification is terminated 13. However, if the desired droplet characteristics specification is not achieved, steps 9 to 12 mentioned above are repeated 14.

It is to be appreciated that the droplet characteristics specification as described in FIG. 2 is the droplet characteristics specification 7 described in FIG. 1.

It will be appreciated that the system (1A; 1B) of the invention provides greater accuracy, precision and much higher speed in the evaluation of emulsion quality and optimum process time prediction compared to manual evaluation. Also, advantageously the system (1A; 1B) of the invention does not require any dilution of the emulsion, which enables the system to be automated.

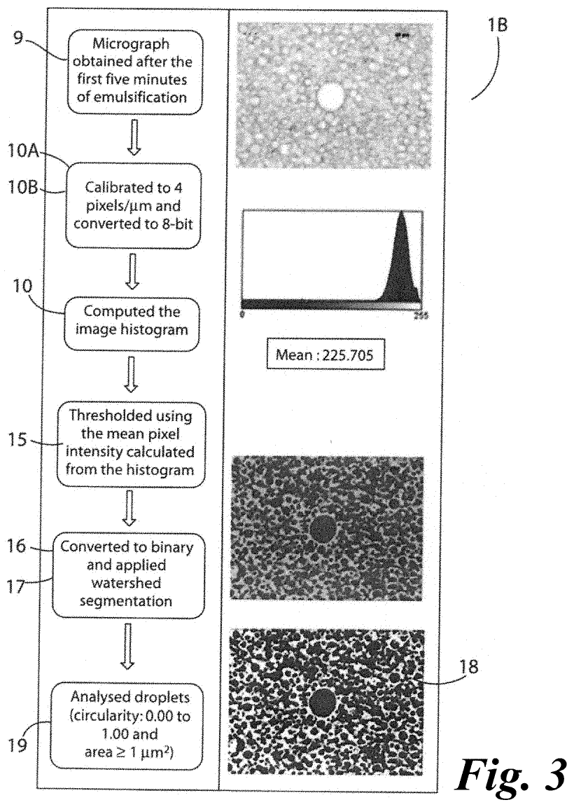

Referring now to FIG. 3 the image processing and droplet detection steps are shown to involve firstly obtaining 9 an image from a pre-set interval during emulsification. In this embodiment the image was obtained at the pre-set interval of the first 5 minutes of emulsification. Calibrating 10A of the images to a scale of 4 pixels/μm, and converting 10B these images to 8-bit greyscale, which provides 2\hat{ }8=256 levels of intensity values for each pixel. In this embodiment the images are shown as micrographs. These micrographs are also referred to as input images. A histogram of the pixel intensity values is then computed 10 for each image and the mean pixel intensity of the histogram is calculated and stored in a variable (not shown). Each image is then thresholded 15 using the calculated mean intensity value and converted 16 to binary. Watershed segmentation 17 is then applied to separate the droplets that touch/overlap each other. Following the above-mentioned image processing an output image 18 is generated ready for analysis. Finally, the resulting output image was analysed 19 for a droplet area range ≥1 μm² and a circularity range of 0.00 to 1.00 to extract the droplet characteristics.

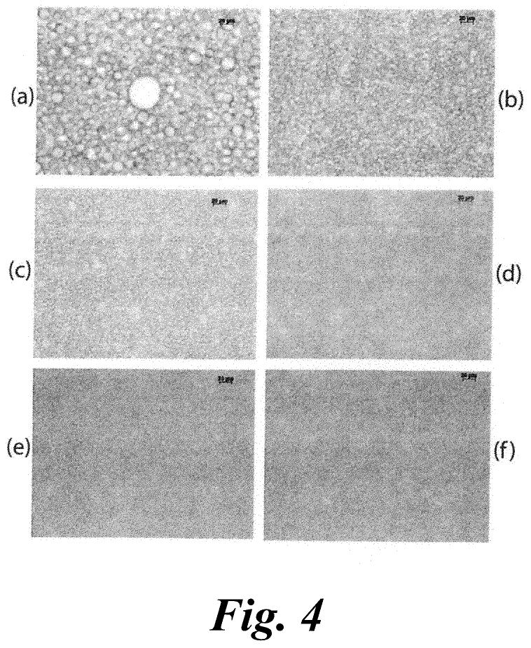

Referring now to FIG. 4, images ((a), (b), (c), (d), (e) and (f)) are obtained at five-minute intervals during emulsification. In this embodiment the images are bright field (BF) 40× micrographs which were saved as TIFF (tagged file format) files (not shown). It is to be appreciated that multiple images (×10) were obtained for each pre-set interval during emulsification. FIG. 4 shows how the droplet characteristics, in particular the size, count and uniformity change over the duration of emulsification. Emulsification was terminated at 30 minutes (f) as further emulsification would result in over processing.

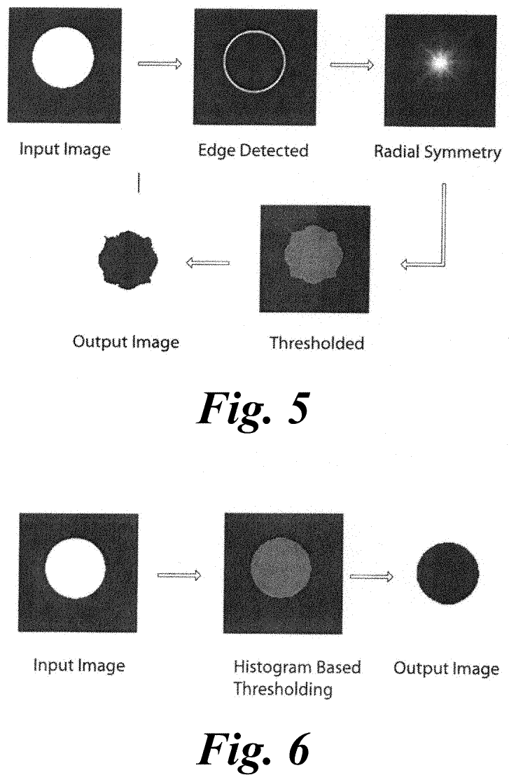

Referring now to FIG. 5, a sample detection of a circular object using an ESF (edge and symmetry filter) algorithm is shown. ESF is a multistage algorithm which operates in two major steps. These are edge detection followed by radial symmetry detection. The edge detection stage includes identification of the edges of all the objects in the input image, centralizing the detected edge points by preserving the local maxima and also minimizing the false edge (noise) detection. The radial symmetry phase makes use of a radius parameter to draw the symmetry of the objects in the image. The ESF treated input image is thresholded to result in an output image ready for analysis.

Referring now to FIG. 6, histogram-based detection of a circular object is shown. An input image, obtained by the imaging device (not shown) undergoes HBT image segmentation as herein described to result in an output image that can be used for analyzing.

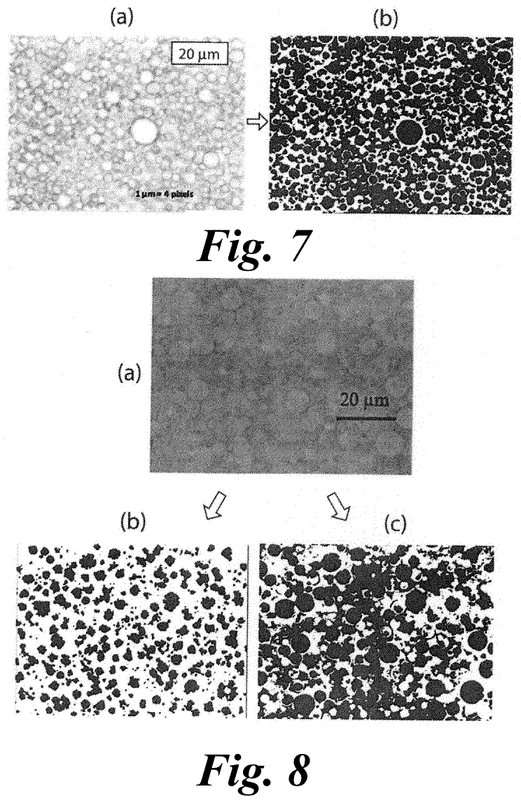

Referring now to FIG. 7, an input image of an emulsion micrograph was obtained after the first five minutes of emulsification. After HBT image segmentation was applied, an output image (b) was produced. Approximately 1,500 to 2,000 droplets were detected from the output image obtained.

Referring now to FIG. 8, a comparison was made of the droplet detection from an emulsion micrograph using both segmentation methods. An input image (a) is processed using one of the image segmentation techniques ESF (b) or HBT (c), resulting in an output image ready for analysis. The HBT output image (c) is shown to provide more clearly defined droplets.

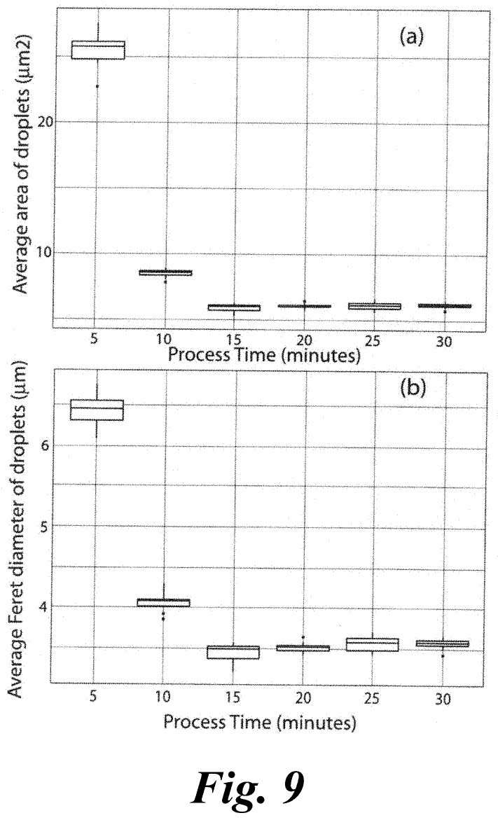

Referring now to FIG. 9, box plots of average area and average Feret diameter of the droplets obtained using edge and symmetry filter are presented. Feret diameter is the longest distance between any two points along the droplet boundary. The average droplet area decreased from around 30 to 5 μm² in the first 15 minutes of the emulsification process and it appears to be varying only slightly for the rest of the processing period. Similarly, the average Feret diameter falls from around 7 to 3 μm in the initial 15 minutes and doesn't appear to vary much after that.

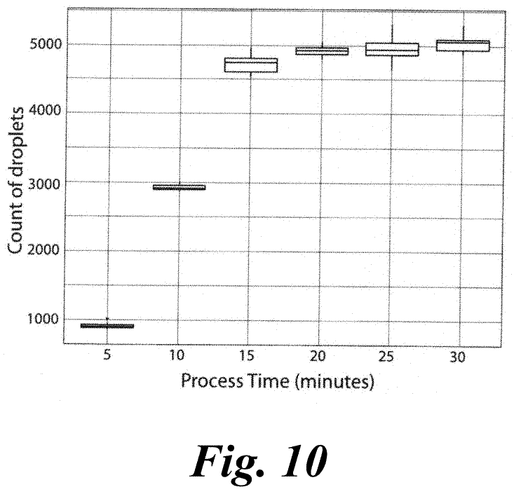

Referring now to FIG. 10, a box plot showing the evolution of droplet count is given. The droplet count seems to increase by 4000 in the first 20 minutes of the emulsification process. Then it varies slightly in the next 5 minutes followed by a steady state.

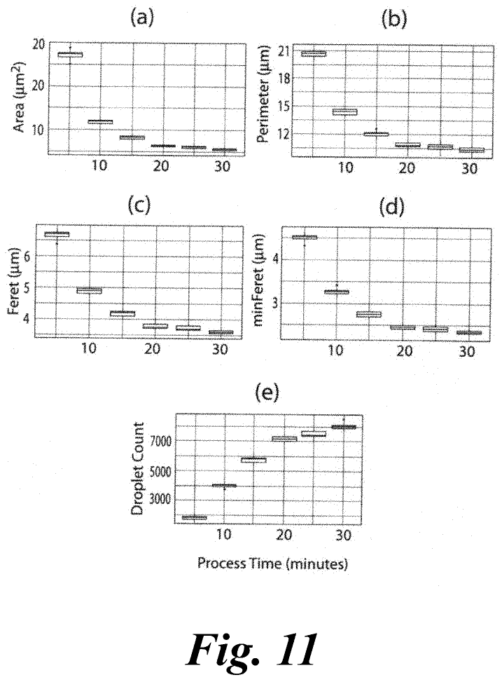

Referring now to FIG. 11 the variation in the droplet characteristics throughout the process was statistically analysed using the box plots presented. Among the 13 characteristics obtained from each droplet, the size features such as area, perimeter, Feret and MinFeret as well as droplet count showed the most significant variation during the emulsification process. The remaining droplet characteristics such as orientation, shape and centroid were considered less informative, as they showed little to no variation throughout the emulsification.

Each box plot, from FIGS. 11(a) to 11(d), represents the mean droplet size obtained from the 10 micrographs at every five minute interval. A sharp decrease was observed in the mean droplet size during the first 10 minutes followed by a progressive decrease throughout the remaining emulsification process. In the last 15 minutes, i.e., from 20 to 30 minutes on the x-axis of FIGS. 11(a) to 11(d), minimal variation was observed in the droplet size, which indicated the process approaching a steady state. The droplet count presented in FIG. 11(e) shows a sharp increase initially followed by minimal variation during the last 10 minutes of the emulsification process.

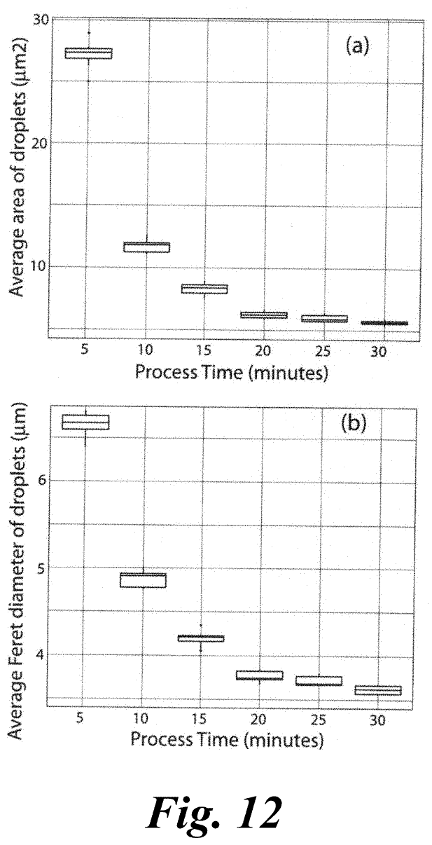

Referring now to FIG. 12, box plots of average area and Feret diameter of the droplets detected at each 5 minutes interval of the emulsification process using histogram-based detection are presented. In the initial 10 minutes, the droplet area decreases dramatically from around 30 to 10 μm² while the Feret diameter also drops from 7 to 5 μm. During the following 20 minutes, the droplet size appears to decrease gradually and attains a steady state towards the end of the emulsification process.

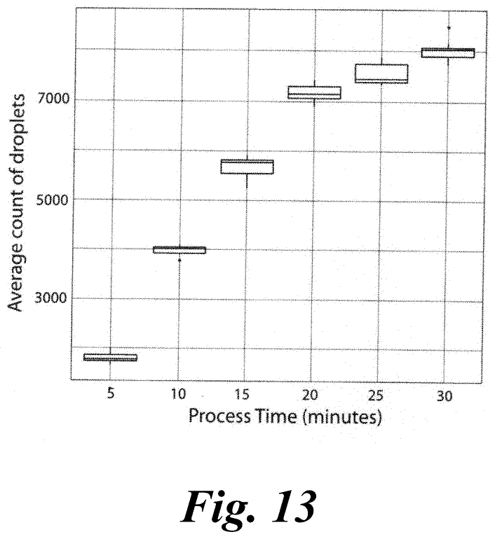

Referring now to FIG. 13, the evolution of droplet count shows a very smooth increase from around 2000 to 8000 droplets during the total 30 minutes of the emulsification process.

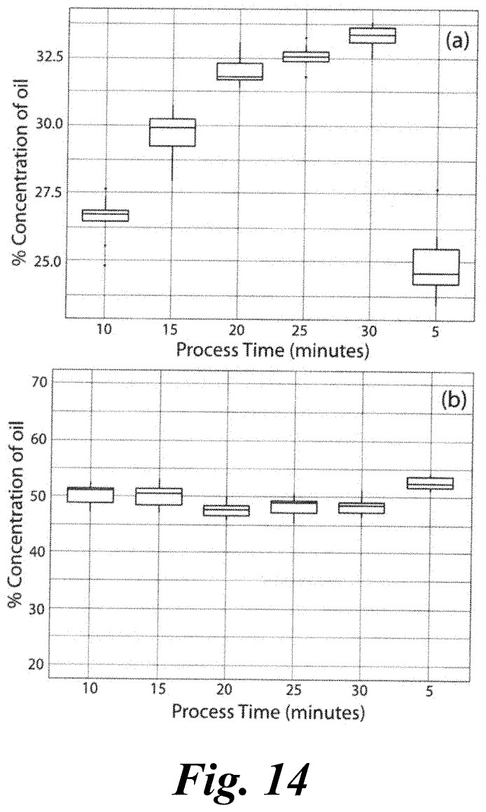

Referring now to FIG. 14, box plots were generated for the concentration of oil obtained using the micrographs from 5 to 30 minutes of emulsification process as shown. The box plot shown in FIG. 14(a), obtained using edge and symmetry, shows inconsistency in the oil concentration throughout the process and the values do not agree with the oil to water ratio of the product. On the other hand, the oil concentration results obtained using the histogram-based method of the invention (FIG. 14(b)) was in close agreement with the emulsion product and showed consistency throughout the process.

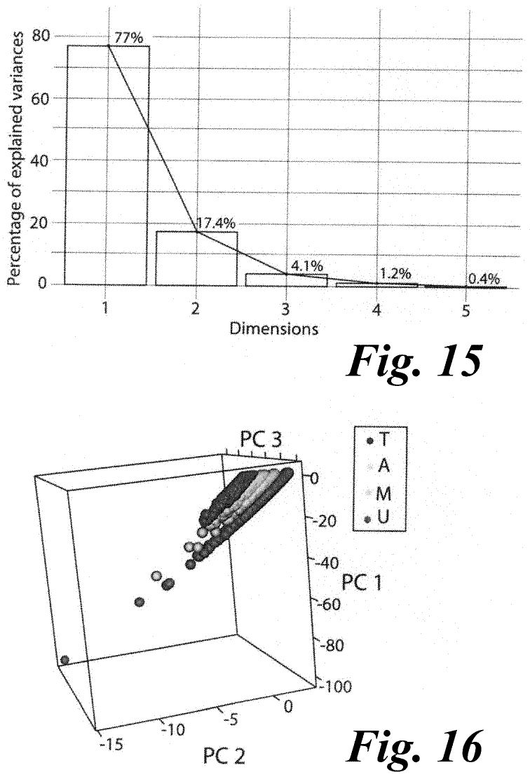

Referring now to FIG. 15, a scree plot of the five principal components (PCs) is shown. The Scree plot is used to select the most significant PCs and shows the percentage of total variance in the data as explained by each PC. The score plots of the first three PCs were also graphed. The PCs, which explained a significant percentage of the variance in the data and were also identified as the most relevant for classification, were selected as the predictor variables to build the supervised classification model using Linear Discriminant Analysis (LDA). The PC-LDA model accuracy was evaluated using stratified 5-fold cross-validation. The first three PCs together explained a cumulative variance of 98.5% (i.e., 77% (of dimension 1) plus 17.4% (of dimension 2) plus 4.1% (of dimension 3)), which was found sufficient to represent a significant proportion of the total variance in the original feature space.

Referring now to FIG. 16, principal component analysis score plots using the first three PCs is shown, categorized into one of the following: ‘U’ (Unacceptable; dark grey circles at bottom), ‘M’ (Marginal; grey circles at second to bottom), ‘A’ (Acceptable; light grey circles at second to top) and ‘T’ (Target; black circles at the top). The droplets obtained from the TAMU categories, represented by the four different colours and relative position, were separated into four clusters along the PC1-PC2 plane as shown. PCA also helped to reduce the dimensionality and the correlation of the original feature space from five correlated variables down to three uncorrelated components. PC1, PC2 and PC3 were selected as the predictor variables for developing the LDA classification model.

It is to be appreciated that the supervised classification model built using the selected PCs of FIGS. 15 and 16 as predictor variables along with Linear Discriminant Analysis (LDA), together known as PC-LDA, forms the basis of the desired droplet specification against which the measured droplet characteristics are compared.

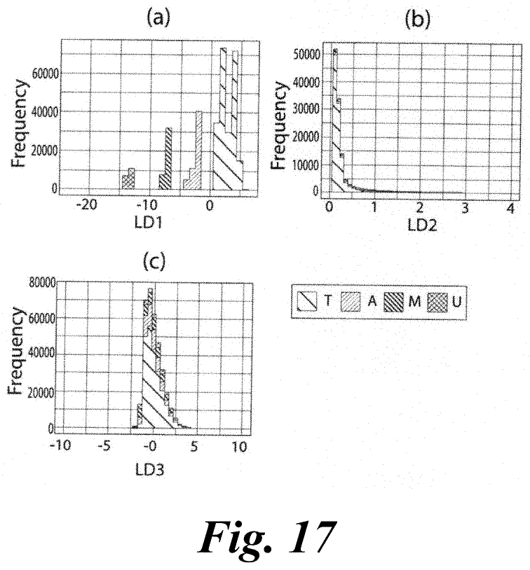

Referring now to FIG. 17, LDA classification is presented by the three discriminant functions a) LD1, b) LD2 and c) LD3. The classification presented by each discriminant function was observed by plotting the histograms shown in FIGS. 8(a) to 8(c). The first discriminant function, LD1, was found to best separate the four TAMU categories. The percentage of classification achieved by each discriminant function is explained by the proportion of trace given by the model, which was 99.86% for LD1.

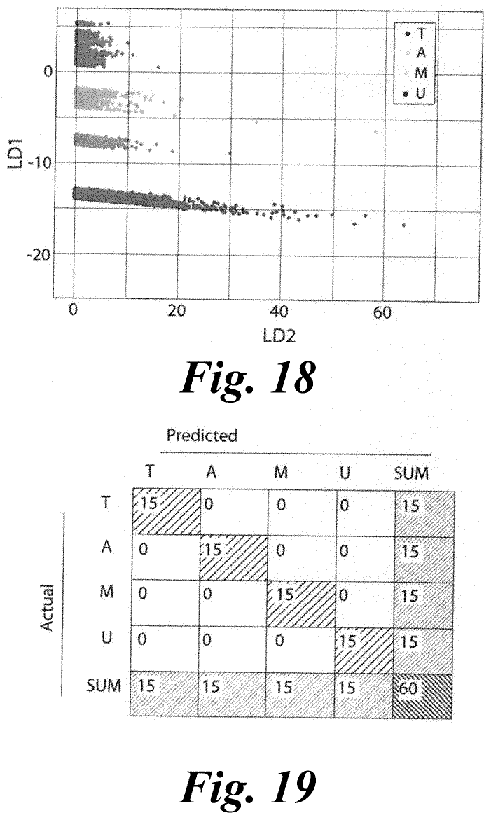

Referring now to FIG. 18, TAMU classification is presented by a two-dimensional scatter plot of LD2 vs LD1 to show the overall separation between the four categories. In descending order, the classification categories are shown as follows: ‘T’ (Target; black circles at top), ‘A’ (Acceptable; light grey circles, second to top), M′ (Marginal; grey circles, second to bottom) and ‘U’ (Unacceptable; dark grey circles at bottom). The scatter plot presents a good classification between the four TAMU categories along the LD1 axis. In summary, the number of canonical variables selected for the PC-LDA model was reduced to one (LD1).

Referring now to FIG. 19, a confusion matrix of PC-LDA Model from 5-fold cross-validation representing the sum of five confusion matrices from the five models is shown. The cells descending diagonally from top left to bottom right (15) represent the correct classification from each category, i.e., the sum of the correct classification in each category obtained from the five models (3×5=15). The cell at the bottom right hand corner (60) represents the total number of correct classification, i.e., the overall sum of the correct classification (12×5=60). In the 5-fold cross-validation, the test sets of micrographs (12 in each model) in all the five folds (models) achieved 100% correct classification of their corresponding droplets.

The examples given below present an investigation of two different image segmentation techniques that were performed to extract various droplet characteristics from images obtained by the imaging device, such as optical micrographs, during emulsification.

### Methodology and Analysis

Methods described below are modelled on typical industrial production of cream-based emulsions.

The emulsion was continuously mixed for 30 minutes using a homogenizer at a tip speed of 25 m/s for the first 15 minutes and at a tip speed of 15 m/s for the last 15 minutes respectively. Microscopic images (micrographs), of an oil in water (o/w) cream emulsion, where acquired at 5-minute intervals from the start to the end of a 30-minute emulsification process. The emulsification process was stopped after 30 minutes, as any further processing was considered as over processing.

It will be appreciated that while a typical o/w emulsion is shown, the apparatus and method would also suitably work with other types of emulsion. While micrographs were obtained as the image of choice, other image types are envisioned to also suitably work in the system herein described. Furthermore, while the method described shows a standard o/w emulsification the system is not to be considered as limited to controlling only o/w emulsions, being suitable for controlling emulsification of other emulsion types.

A Zeiss Microscope Axio imager Atm was employed to obtain bright field micrographs of 40× magnification under standard illumination settings from each sample and were saved as TIFF (tagged file format) files. An example of the images obtained can be seen in FIG. 4. Two different automated image segmentation techniques were developed in Fiji version 1.51 h to identify the region of interest, which is the oil droplet, in the micrographs. Subsequently, the droplet characteristics were extracted using both the techniques. Fiji is an extended version of ImageJ.

A macro, user-defined program to execute specific tasks, was programmed in Fiji to execute an image segmentation procedure.

The edge and symmetry-based image segmentation steps dynamically in the following order. First the micrographs were calibrated to a micrometer scale such as 4 pixels/μm. The images were then converted to 8-bit in order to facilitate further processing. Edge and Symmetry filter (ESF) was applied to detect the oil droplets in the images. The filtered images were auto-thresholded through the red channel to enhance the detected droplets. The images were then converted into binary and ‘watershed segmentation’ was applied separating the droplets that touch/overlap each other.

Alternatively, an image segmentation procedure based on producing a histogram of the distribution of gray values (pixel intensity) in the region of interest.

Calibration of the micrographs to a scale of 4 pixels/μm and conversion to 8-bit greyscale were then performed which provides 2\hat{ }8=256 levels of intensity values for each pixel.

This was followed by histogram-based image segmentation, which was executed. First a histogram was computed from all of the pixels in the images for each image. The mean pixel intensity of the histogram was calculated and stored in a variable. The images were then thresholded using the calculated mean intensity value. Converted to binary and watershed segmentation was applied to separate the droplets that touch/overlap each other. Finally, each micrograph was analysed for a droplet area range ≥1 μm² and a circularity range of 0.00 to 1.00 to extract the droplet characteristics. The minimum size settings can be changed to detect droplets down to nanometres.

It is to be appreciated that such calibration and conversion may form part of the initial steps of the macro.

The droplets detected using one of the two methods of image segmentation were then analyzed using the ‘Analyze Particles’ functionality in Fiji. The purpose of this image analysis was to extract the characteristics of the detected droplets for a user-specified range of droplet area (μm²) and circularity. A diverse set of characteristics was extracted for each droplet such as size, shape, orientation, centroid, solidity etc.

Thirteen characteristics were extracted for each droplet from the 60 micrographs.

These are the following:
Size features: area (μm²), perimeter (μm), maximum Feret diameter (Feret in μm), minimum Feret diameter (MinFeret in μm)
Centroid coordinates: X & Y
Starting coordinates of Feret diameter: FeretX & FeretY
Orientation characteristic: Feret angle.
Shape features: Circularity, roundness, aspect ratio and solidity.

The droplet characteristics were automatically exported, by the macro, into a CSV file in the user-specified directory.

It is to be appreciated that such analysis may form part of the final steps of the macro.

Statistical analysis of the droplet characteristics was performed in RStudio, version 1.1.383 for all the micrographs, which is an integrated development environment for programming using the R language. R 3.4 is the version used in this study. The mean values of the droplet characteristics were calculated and box plots were generated to identify the characteristics that varied significantly over the emulsification period. The R data visualization package ggplot2 was used to create the plots. The variation in the mean and median of each characteristic and also the correlation between them were also investigated. A reduced set of droplet characteristics, deemed suitable, were finalized as the input feature space for the classification of micrographs. The evolution of average droplet size in terms of area (μm²), perimeter (μm), minimum Feret diameter (MinFeret in μm) and maximum Feret diameter (Feret in μm) and the evolution of droplet count at each 5-minute processing interval were studied in detail. The oil concentration, i.e., the % of area occupied by the droplets, was also determined for each micrograph.

The total set of 60 micrographs were categorised manually into four groups named Unacceptable (U), Marginal (M), Acceptable (A) and Target (T) by micrograph analysts from industry. An unsupervised cluster analysis of the droplet characteristics was performed using PCA to observe the patterns in the data. The PCA technique also helped to reduce the dimensionality of the data and to obtain a set of uncorrelated components.

The variation in the droplet characteristics throughout the process was statistically analysed using the box plots. Among the 13 characteristics obtained from each droplet, only the size features such as area, perimeter, Feret and MinFeret as well as droplet count showed significant variation during the emulsification process. The remaining droplet characteristics such as orientation, shape and centroid were not considered relevant, as they showed no variation throughout the emulsification process.

The overall variation in the mean droplet characteristics during the whole 30-minute process can be summarised as follows:
Area decreased from 27.1 to 5.6 μm².
Perimeter decreased from 20.6 to 10.3 μm.
Maximum Feret diameter (Feret) dropped from 6.7 to 3.6 μm.
Minimum Feret diameter (MinFeret) dropped from 4.5 to 2.3 μm.
Droplet count increased from 1,500 to 8,500.

On the basis of the variation presented by the mean droplet size characteristics as well as the droplet count, including the knowledge of process experts, the 60 micrographs were manually grouped into four quality-based categories referred to as TAMU. The processing intervals to which the categories belonged to are shown in Table 1.

TABLE 1
Micrograph categories named from 5 to 30 minutes based on the
variation in the droplet characteristics.

| Process time | Category |
|---|---|
| 5 minutes | Unacceptable |
| 10 minutes | Marginal |
| 15 minutes | Acceptable |
| 20, 25, 30 minutes | Target |

The 10 sample micrographs obtained from the first five minutes were categorised as ‘Unacceptable’ (U), another 10 micrographs obtained after 10 minutes of emulsification were labelled as ‘Marginal’ (M), the next 10 obtained after 15 minutes, in total, were categorised as ‘Acceptable’ (A) and the 30 micrographs acquired after 20 minutes until the end of the process were grouped as ‘Target’ (T).

The five droplet characteristics, area, perimeter, Feret, MinFeret and count, were selected as the input features for building the classification model. A correlation matrix of the droplet characteristics was obtained and is presented in Table 2.

TABLE 2
Correlation matrix of the droplet size characteristics selected as
the input feature space for the classification model.

| | Area | Perimeter | Feret | MinFeret |
|---|---|---|---|---|
| Area | 1.00 | 0.85 | 0.84 | 0.87 |
| Perimeter | 0.85 | 1.00 | 0.98 | 0.96 |
| Feret | 0.84 | 0.98 | 1.00 | 0.94 |
| MinFeret | 0.87 | 0.96 | 0.94 | 1.00 |

The droplet size characteristics were found to be highly correlated (r=0.84 to 1.00). The next major step in the analysis was to covert the correlated feature space into a reduced set of uncorrelated components. This was followed by an unsupervised cluster analysis of the components to distinguish the TAMU patterns identified by the industrial experts.

PCA was performed to transform the five variable feature space into an equal set of uncorrelated principal components (PCs). An unsupervised clustering pattern of the four response categories (TAMU) was also explored using the score plots of the first three PCs.

A PC-LDA model was developed as a linear combination of the first three principal components, PC1, PC2 and PC3. The number of predictive discriminant functions derived from the model is calculated as the minimum of G−1 and p, where G is the number of response categories (i.e., four) and p is the number of predictor variables (i.e., three). In the current case, the value of p and G−1 are equal and therefore, the PC-LDA model resulted in three discriminant functions, LD1, LD2 and LD3, as given by Equations 1, 2 and 3. The model formula is represented by Equation 4.

LD1=1.03PC1−0.31PC2−0.35PC3 Equation (1)

LD2=−4.68PC1−0.29PC2−0.35PC3 Equation (2)

LD3=0.13PC1+1.70PC2−1.46PC3 Equation (3)

Category˜LD1+LD2+LD3 Equation (4)

The discriminant functions, as given by the above-mentioned equations, were then used to separate the four TAMU categories, the results of which are presented and discussed under FIGS. 17(a), 17(b) and 17(c).

A stratified 5-fold cross-validation was performed to evaluate the classification accuracy of the PC-LDA model. 10 micrographs from each category were selected for the cross-validation. In each fold, the micrographs were randomly split into 70% for training and the remaining 30% for testing. Five models were created in such a way that each model consisted of a training set of 28 micrographs (seven from each category) and a test set of 12 micrographs (three from each category) respectively. For each model, the classification score was recorded and a confusion matrix was created.

The models were further validated using a set of six micrographs (two from ‘U’ category, two from ‘M’, one from ‘A’ and one from ‘T’ respectively). These micrographs were obtained from a laboratory, in a different country, under varying illumination settings. All the five models successfully classified five out of the six micrographs into the correct category except the Target (T) one, which was classified as Acceptable (A). This gives an overall accuracy of 83%.

The droplets detected from a sample micrograph acquired after the initial five minutes of processing. Similarly, the droplet characteristics of all the 60 micrographs were extracted automatically in an iterative loop by the macro.

The edge and symmetry detection technique highly depends on well-defined droplet borders and also on the radius parameter of the algorithm specified by the user. As the droplet size decreases with emulsification, it is difficult for the user to identify an accurate value for the droplet radius. This limits the use of this technique. Multiple detections will be required for a single micrograph, using a set range of radii, to detect droplets from a varying size distribution, which is not feasible in terms of time and accuracy.

Such edge detection techniques have proved less successful in the image analysis of highly concentrated emulsions with dispersed phase fraction greater than 10 to 15%.

The existing image segmentation techniques, as described in the literature, for droplet detection from emulsion micrographs are mainly border-based edge detection methods. Such techniques have demonstrated potential only in the case of high-quality images, droplets with pronounced borders, less overlap and also in emulsions with low dispersed phase fraction ≤15%. By contrast, the histogram-based technique (HBT) as used in the present invention is capable of detecting both bigger and smaller droplets thus providing a smooth evolution of droplet size and count. The droplet size decreases significantly during the last minutes of processing and they appear as texture rather than discrete droplets. The histogram-based approach demonstrates proficiency in detecting those droplets.

The HBT method presented good detection of both bigger and smaller droplets. An R function was written to read the droplet characteristics of each micrograph from the CSV file (saved by the macro) in a sequential loop and create box plots showing their evolution over emulsion processing time. A plugin was developed in Java language, as a soft sensor, to integrate the Fiji macro with the R function. For these reasons, HBT is the preferred image segmentation technique for use in the present invention.

The machine vision techniques ESF and HBT were compared by analyzing the oil concentration obtained using the image analysis.

The HBT demonstrates potential in assessing the evolution of droplet characteristics during emulsification until the desired characteristics are achieved. Therefore, the technique can be implemented as an automated tool in chemical industries to predict and identify the optimum process time. This can contribute to eliminating over-processing and associated resources that can be regarded as surplus to production requirements.

The system of the invention can entirely automate, in real-time, the quality assessment of the emulsion. It is possible to implement the system of the invention by integrating the machine vision technique with real-time imaging (an endoscope coupled with a CCD camera). The system of the invention can be applied as a soft sensor, for in-situ process monitoring, to provide real-time feedback on emulsion quality.

The competence of edge & symmetry and histogram-based machine vision techniques, applied on optical micrographs, in the valuation of droplet characteristics during emulsification is hereinabove shown. Droplet area, Feret diameter and count are the characteristics identified as the product quality indicators.

The edge & symmetry technique is less advantageous, as it does not lend as well to full automation. This is due to the need to recalibrate the radius parameter as droplet sizes change. On the other hand, the histogram-based approach of the present invention is fully automated. The histogram-based image segmentation has demonstrated significant potential in the detection of droplets and their corresponding characteristics. The technique has provided a progressive evolution of decreasing droplet size and increasing droplet count. The oil concentration results obtained using the histogram-based approach is in good agreement with the studied emulsion. The technique is capable of identifying the equilibrium point at the end of the industrial process. The system of the invention avoids the over-processing of emulsions, leading to smart and sustainable manufacturing practices.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described which may be varied in both construction and detail within the scope of the appended claims.

1. An apparatus for controlling emulsification of at least two immiscible fluids comprising:
an imaging device configured to obtain an image of at least one droplet at pre-set intervals between a start and an end of the emulsification;
an image processor configured to detect and measure at least one droplet characteristic;
means for analyzing the measured droplet characteristics;
means for comparing the measured droplet characteristics with a desired droplet characteristics specification; and
a control switch configured to terminate the emulsification when a signal is received that said desired droplet characteristics are achieved.

2. The apparatus as claimed in claim 1, wherein the imaging device is adapted for mounting in situ where the emulsification process is taking place.

3. The apparatus as claimed in claim 1, wherein the imaging device is an optical imaging device.

4. The apparatus as claimed in claim 1, wherein the image obtained by the imaging device is a micrograph.

5. The apparatus as claimed in claim 4, wherein the micrograph is calibrated to a micrometer scale.

6. The apparatus as claimed in claim 1, wherein the image processor is configured to detect the droplet from the pixels in the image.

7. The apparatus as claimed in claim 1, wherein the image processor configured to detect the droplets comprises a means for image segmentation.

8. The apparatus as claimed in claim 7, wherein the means for image segmentation is selected from one of an edge and symmetry filter and histogram-based technique.

9. The apparatus as claimed in claim 1, wherein the image processor configured to detect at least one of the droplet characteristics comprises means to prepare a graphical representation from the pixels in the images.

10. The apparatus as claimed in claim 1, wherein the graphical representation is selected from one of an edge and symmetry graphic and a histogram.

11. (canceled)

12. The apparatus as claimed in claim 1, wherein the apparatus comprises means to threshold the image.

13. The apparatus as claimed in claim 12, wherein the apparatus comprises means to convert the thresholded images to binary.

14. The apparatus as claimed in claim 1, wherein the apparatus comprises means to apply watershed segmentation to the image.

15. The apparatus as claimed in claim 1, wherein the apparatus comprises means to convert the image to 8-bit.

16. The apparatus as claimed in claim 1, wherein the means for comparing the measured droplet characteristics obtained from the micrograph with the desired droplet characteristics specification comprises a supervised classification model.

17. (canceled)

18. (canceled)

19. (canceled)

20. The apparatus as claimed in claim 1, wherein the apparatus comprises a means for alerting the emulsion quality category of the images obtained by the image processor corresponding to a quality of the emulsion at the pre-set intervals between the start and the end of emulsification.

21. (canceled)

22. (canceled)

23. (canceled)

24. A method for controlling an emulsification process including the steps:
acquiring images of at least one droplet at pre-set intervals between a start and an end of the emulsification process;
detecting and measuring at least one droplet characteristic from the acquired images;
analyzing the measured droplet characteristics;
comparing the measured droplet characteristics with a desired droplet characteristic specification; and
terminating the emulsification process when said desired droplet characteristic is achieved.

25. (canceled)

26. (canceled)

27. The method as claimed in claim 24, wherein each image obtained by imaging device is a micrograph and the method comprises calibrating the micrograph to a micrometer scale.

28. (canceled)

29. (canceled)

30. (canceled)

31. (canceled)

32. (canceled)

33. (canceled)

34. The method as claimed in claim 24, wherein the method comprises categorizing the emulsion into one of several pre-defined emulsion quality categories.

35. The method as claimed in claim 34, wherein the method comprises processing data on the emulsion quality category obtained from the pre-set intervals between the start and the end of emulsification to determine when the desired droplet characteristics are achieved.

36. (canceled)
